(19) United States
(12) Patent Application Publication
(10) Pub. No.: US 2024/0180042 A1
(43) Pub. Date: May 30, 2024

(54) Earth-abundant dopants for piezoelectric enhancement in wurtzite crystals

(71) Applicant: National Technology & Engineering Solutions of Sandia, LLC, Albuquerque, NM (US)

(72) Inventors: Remi Philippe Michel Dingreville, Corrales, NM (US); Jacob Kyle Startt, Albuquerque, NM (US); Nathan Jackson, Albuquerque, NM (US)

(73) Assignee: University of New Mexico Rainforest Innovations, Albuquerque, NM (US)

(21) Appl. No.: 18/517,304

(22) Filed: Nov. 22, 2023

Related U.S. Application Data
(60) Provisional application No. 63/428,286, filed on Nov. 28, 2022.

(51) Int. Cl. H10N 30/85; H10N 30/092

(52) U.S. Cl. CPC H10N 30/852; H10N 30/092

(57) ABSTRACT

Doped-AlN piezoelectric materials are advantageous because they are far more compatible with complementary metal oxide semiconductor (CMOS) materials and they maintain both piezoelectric and thermodynamic stability up to very high temperatures, compared to PZT. Ab-initio calculations and targeted experimentation have identified alternative, earth-abundant, dopants for AlN from the periodic table d-block. In particular, group IVB elements, titanium (Ti), zirconium (Zr), and hafnium (Hf) induce large piezoelectric enhancements comparable to rare-earth dopants, such as Sc. This improvement is due to shifts in the sublattice atomic structure and changes in the local charge states. This invention provides a highly accessible and affordable path for technological adaptation of AlN-based piezoelectrics for sustainable, next-generation electronics.

### CROSS-REFERENCE TO RELATED APPLICATION

This application claims the benefit of U.S. Provisional Application No. 63/428,296, filed Nov. 28, 2022, which is incorporated herein by reference.

### STATEMENT OF GOVERNMENT INTEREST

This invention was made with Government support under Contract No. DE-NA0003525 awarded by the United States Department of Energy/National Nuclear Security Administration. The Government has certain rights in the invention.

### STATEMENT REGARDING PRIOR DISCLOSURES BY THE INVENTOR OR A JOINT INVENTOR

The following disclosure is submitted under 35 U.S.C. 102(b)(1)(A): Jacob Startt, Mohammed Quazi, Pallavi Sharma, Irma Vazquez, Aseem Poudyal, Nathan Jackson, and Remi Dingreville, “Unlocking AlN Piezoelectric Performance with Earth-Abundant Dopants,” Advanced Electronic Materials 9, 2201187 (2023). The subject matter of this disclosure was conceived of or invented by the inventors named in this application.

### BACKGROUND OF THE INVENTION

The discovery of new affordable, easy-to-produce, and chemically stable piezoelectric materials would enable a wide range of emerging functionalities related to actuation (e.g., for nano- and micro-positioners, ultrasound medical devices, acoustic wave resonators, and 5G and beyond communications) and sensing (e.g., in electroacoustic and immunosensors, aircraft engines, instrument pick-ups, microphones, touch-sensitive screens, and activity monitors in implanted or smart wearable devices). See C. Tian and P. Yan, Mechatronics 65, 102321 (2020); A. Cafarelli et al., ACS Nano 15, 11066 (2021); J. Lim et al., Biochem. Biophys. Rep. 30, 101265 (2022); S. V. Krishnaswamy et al., IEEE Symposium on Ultrasonics 1, 529 (1990); W. Wang et al., Appl. Phys. Lett. 105, 133502 (2014); Y. Song et al., ACS Appl. Mater. Interfaces 13, 19031 (2021); M. Pohanka, Materials 11, 448 (2018); T. Manzaneque et al., Sens. Actuators A Phys. 220, 305 (2014); H. Elahi et al., Integr. Ferroelectr. 211, 25 (2020); G. Piana et al., Meccanica 51, 2797 (2016); S. Horowitz et al., J. Acoust. Soc. Am. 122, 3428 (2007); S. Goncalves et al., ACS Appl. Electron. Mater. 1, 1678 (2019); A. H. Anwer et al., Sensors 22, 4460 (2022); T. Okano et al., J. Signal Process. Syst. 91, 1053 (2019); N. Sezer and M. Koc, Nano Energy 80, 105567 (2021); and B. Shi et al., Adv. Mater. 30, 1801511 (2018). Today, the most widely used and studied piezoelectric materials are ferroelectric ceramics, such as Pb(ZrₓTi₁₋ₓ)O₃ (lead zirconate titanate, PZT), BaTiO₃ (barium titanate) and other perovskite titanates. See M. Safaei et al., Smart Mater. Struct. 28, 113001 (2019); H. Jaffe, J. Am. Ceram. Soc. 41, 494 (1958); S. Trolier-Mckinstry and P. Muralt, J. Electroceramics 12, 7 (2004); and P. Muralt, J. Micromech. Microeng. 10, 136 (2000). While these materials exhibit good piezoelectric properties, they are typically incompatible with complementary metal-oxide-semiconductor (CMOS) components and their low Curie temperatures (˜300 to 400° C.) cause a dramatic and rapid drop in their ferroelectric and piezoelectric properties with increasing temperature. See Z. Gubinyi et al., J. Electroceramics 20, 95 (2008); and P. V. Balachandran et al., Phys. Rev. B 93, 144111 (2016). In addition, PZT is highly toxic and increasingly so at high temperatures due to a high volatility. See P. K. Panda, J. Mater. Sci. 44, 5049 (2009). In contrast, aluminum nitride (AlN) compounds have both high melting temperatures (˜2500° C.) and high Curie temperatures (˜1150° C.) and thus retain their piezoelectric properties at elevated temperatures. See A. Abid et al., J. Mater. Sci. 21, 1301 (1986); and M.-A. Dubois and P. Muralt, Appl. Phys. Lett. 74, 3032 (1999). Such high-temperature stability gives AlN a distinct advantage over other materials for a broad range of applications. See G. Piazza et al., MRS Bull. 37, 1051 (2012). However, undoped AlN thin films have a poor natural piezoelectric response in comparison to other piezoelectrics, like PZT. See C. M. Lueng et al., J. Appl. Phys. 88, 5360 (2000).

Although the modulus of piezoelectricity of pure AlN thin films is low (d₃₃=5.5 pCN⁻¹), it can be increased by the addition of one or more doping elements. Most notably, Akiyama et al. showed that the d₃₃ modulus in sputter-deposited AlN thin films can be enhanced by nearly 500% when doped with scandium (Sc). See M. Akiyama et al., Adv. Mater. 21, 593 (2009). Later investigations found that other rare-earths, such as yttrium (Y) and ytterbium (Yb), can also induce similar piezoelectric enhancements in AlN. See C. Tholander et al., Phys. Rev. B 87, 094107 (2013); K. Hirata et al., ACS Omega 4, 15081 (2019); and P. M. Mayrhofer et al., Acta Mater. 100, 81 (2015). Unfortunately, two factors limit the integration of these dopants into commercial process flow: rare-earth sputtering targets such as Sc and Yb are expensive, and fabricating stable films with increasing concentrations of these doped elements has proven to be challenging. Thus, there is a urgent economical need for alternative, earth-abundant, and affordable dopants capable of inducing comparable piezoelectric enhancements in AlN films. In response, several research groups have started to investigate the effects of other doping elements, such as titanium (Ti), zirconium (Zr), hafnium (Hf), niobium (Nb), and tantalum (Ta). These groups found that some of these elements can yield improvements on the performance of AlN-based piezoelectrics. See N. Farrer and L. Bellaiche, Phys. Rev. B 66, 201203 (2002); A. Alsaad and A. Ahmad, Eur. Phys. J. B 65, 65 (2008); M. Akiyama et al., Adv. Mater. 21, 593 (2009); J. T. Luo et al., J. Phys. Appl. Phys. 42, 235406 (2009); F. Tasnadi et al., Appl. Phys. Lett. 94, 151911 (2009); F. Tasnadi et al., Phys. Rev. Lett. 104, 137601 (2010); E. Iborra et al., in 2012 IEEE Int. Ultrason. Symp., pp. 2734-2737 (2012); E. Iborra et al., in 2013 Joint Eur. Frequency and Time Forum & Int. Frequency Control Symposium (EFTF/IFC), pp. 262-265 (2013); H. Liu et al., Appl. Surf. Sci. 270, 225 (2013); C. Tholander et al., Phys. Rev. B 87, 094107 (2013); Y. Iwazaki et al., Appl. Phys. Express 8, 061501(2015); C. Tholander et al., Phys. Rev. B 92, 174119 (2015); M. Uehara et al., Appl. Phys. Lett. 111, 112901 (2017); K. Hirata et al., ACS Omega 4, 15081 (2019); M. Noor-A-Alam et al., ACS Appl. Mater. Interfaces 13, 944 (2020); H. Fiedler et al., Appl. Phys. Lett. 118, 012108 (2021); and T. Terada et al., Jpn. J. Appl. Phys. 60, SFFB08 (2021). However, despite these advances, only a limited portion of the periodic table and dopant composition space has been explored. These prior studies were largely focused on the effects of co-doping transition metals with light metals, often with no experimental validation, and were seemingly limited in the compositional range explored or they employed non-standard fabrication techniques for which the concentration of doped elements was exceedingly small. See, e.g., E. Iborra et al., in 2012 IEEE Int. Ultrason. Symp., pp. 2734-2737 (2012); and H. Fiedler et al., Appl. Phys. Lett. 118, 012108 (2021).

### SUMMARY OF THE INVENTION

The present invention is directed to a piezoelectric material having a wurtzite crystal structure doped with one or more group IVB or VB transition metal dopants. For example, the wurtzite crystal can comprise AlN, GaN, or ZnO. For example, the piezoelectric material can comprise TMₓAl₁₋ₓN, wherein TM is a group IVB or VB transition metal element and wherein 0.16≤x≤0.5.

As an example, elements from group IVB (Ti, Zr, and Hf), and, less dramatically, group VB (Nb and Ta) enhance piezoelectricity when doped in the w-AlN structure. In particular, the enhancements observed for group IVB doped-AlN structures are comparable to those observed in Sc- and Y-doped AlN thin films, with Zr and Hf yielding 570% and 607% increases in the d₃₃ modulus at (Zr, Hf)_{0.5}Al_{0.5}N and Ti yielding a maximum 345% increase for Ti_{0.33}Al_{0.67}N. Notably, these group IVB transition metal dopants represent economically accessible, earth-abundant alternatives to the more expensive and difficult to obtain Sc, Y, and Yb rare-earth dopants. The methodology and descriptors (i.e., c/a, u, and Bader charges) can also be extended to identify and optimize piezoelectric enhancements in further doped-AlN films in addition to other wurtzite-based piezoelectric films, such as GaN or ZnO. This invention enables easier and more economical integration of highly desirable piezoelectric AlN thin film materials within nano- and microelectromechanical systems which can be CMOS compatible, non-toxic, and stable at high temperatures.

### BRIEF DESCRIPTION OF THE DRAWINGS

The detailed description will refer to the following drawings, wherein like elements are referred to by like numbers.

FIG. 1 is a diagram illustrating the piezoelectric effects of doping wurtzite when AlN is doped with a transition metals. The figure depicts the change in the d₃₃ piezoelectric modulus when AlN is doped with a transition metal (TM) when increasing the dopant concentrations (successive columns). The color indicates the relative sensitivity of the d₃₃ modulus to doping with respect to that transition metal element: darker purple color signifies a strengthening of the modulus, while darker turquoise color signifies a weakening of the modulus. Sensitivities were calculated as the change in d₃₃ as a function of dopant concentration. Gray colored panels showing an ‘n/a’ denote when a transition metal was not modeled (Mn, Ru, Pd, Cd, La, Re, and Ir).

FIGS. 2A-2H illustrate the piezoelectric effects of doping wurtzite AlN structures with the group IVB elements Ti, Zr, and Hf. FIG. 2A shows ab-initio calculations of the d₃₃ piezoelectric modulus in AlN is a function of doping with Ti, Zr or Hf transition metals. FIG. 3B is a graph showing the e₃₃ piezoelectric coefficient as a function of dopant concentration. FIG. 3C is a graph showing the c₃₃ elastic coefficient as a function of dopant concentration. The piezoelectric enhancement is due both to an increase in the underlying e₃₃ piezoelectric coefficient and decrease in the c₃₃ elastic coefficient with dopant concentration. FIG. 3D is a graph showing a relatively minor change in the Born effective charges Z*₃₃ of the cation-planes with doping, suggesting that the enhancements stem not just from changes to the electronic structure and charge states but also from structural features. FIG. 2E is a graph showing a reduction in the c/a lattice constant ratio with doping. FIG. 2F shows the w-AlN crystal structure. FIG. 2G shows the h-AlN structure. The increase in piezoelectricity is facilitated by a partial structural transition from w-AlN to the h-AlN structure. FIG. 2H is a graph that quantifies the shifts in the spacing of cation and anion planes in the [001] direction (measured through the internal parameter u).

FIG. 3A shows energy-dispersive X-ray spectroscopy (EDS) of the measured Ti concentrations (as at % of the whole sample) as a function of Ti RF sputtering power (Al pulsed DC sputtering power was held constant) of the eight fabricated thin films. FIG. 3B shows X-ray diffraction (XRD) analysis indicating that the w-AlN structure is predominantly present in all eight films, while at higher Ti concentrations, peaks belong to rock salt TiN also become visible. FIG. 3C shows a direct comparison of the measured experimental d₃₃ piezoelectric moduli for all eight films, showing agreement with the DFT-predicted moduli. The module in this figure are normalized with respect to the pure, undoped AlN piezoelectric modulus to show the performance improvement of the Ti-doped films.

FIGS. 4A-4F illustrate relationships amongst the piezoelectric moduli and their contributing components in transition metal doped AlN. Ti-, Zr-, and Hf-containing structures are represented respectively by circles, triangles, and squares., while all other structures described in the piezoelectric AlN literature (including those with Sc and Y) are represented by hollow circles. Undoped AlN is marked by the black upside-down triangles. FIG. 4A is a graph of d₃₃ piezoelectric modulus versus e₃₃ piezoelectric coefficient for AlN-based piezoelectrics. FIG. 4B is a graph of d₃₃ piezoelectric modulus versus c₃₃ elastic coefficient. The d₃₃ modulus is highest in compounds which exhibit both the highest e₃₃ piezoelectric coefficients and the lowest c₃₃ elastic coefficients. FIG. 4C shows the strong synergistic relationship between the e₃₃ and c₃₃ coefficients. FIG. 4D is a graph of d₃₃ piezoelectric modulus versus c/a ratio for AlN-based piezoelectrics. FIG. 4E is a graph of d₃₃ piezoelectric modulus versus internal u parameter. Piezoelectricity is observed to increase only in compounds where the c/a ratio decreases and the internal u parameter increases. FIG. 4F shows that these structural features are strongly correlated and can be used to quantify the transition from w-AlN to h-AlN structure. Compounds which have fully transitioned into the h-AlN structure are outlined with boxes in FIGS. 4D-4F.

FIGS. 5A-5F illustrate the relationship of electronic structure and piezoelectricity. Ti-, Zr-, and Hf-containing structures are represented respectively by circles, triangles, and squares, while all other structures (including those with Sc and Y) are represented with hollow circles. Pure AlN is marked by a black upside-down triangle. FIGS. 5A, 5B, and 5C show d₃₃ piezoelectric modulus, e₃₃ piezoelectric coefficient, and c₃₃ elastic coefficient as a function of a dopant's d-shell electrons. Dopant atoms with the fewest d-shell valence electrons exhibit the greatest increases in d₃₃ and e₃₃, and decreases in c₃₃. These transition metals are the most easily ionized in the vicinity of the negatively charged N-sublattice as evidenced by the Z*₃₃ Born effective charges, as shown in FIG. 5D. Simple atomic charges, as counted through a Bader partitioning scheme, shown in FIGS. 5E and 5F, can serve as an adequate and easy to obtain alternative indicators. Compounds which have fully transitioned into the h-AlN phase are outlined with boxes.

### DETAILED DESCRIPTION OF THE INVENTION

Piezoelectricity arises when non-centro-symmetric structures, (i.e., those which lack an inversion center) are strained and an electric dipole moment is formed. A piezoelectric potential can be created in any semiconductor crystal having non central symmetry, such as the Group III-V and II-VI materials, due to polarization of ions under applied stress and strain. This property is common to wurtzite crystal structures, including GaN, AlN and ZnO.

In hexagonal wurtzite structures, like that of w-AlN, this piezoelectric polarization (Pz) is accompanied by an already present spontaneous polarization (Ps), resulting from the stacking and coupling of cation and anion planes along the direction of the c-axis. As such, wurtzite thin films are grown epitaxially, so that the c-axis (and thus direction of piezoelectric polarization) is perpendicular to the film surface. In these systems, the piezoelectric response (i.e., pico-Coulomb of charge per Newton of force, pCN⁻¹) along the c-axis is quantified via the piezoelectric d₃₃ modulus. This quantity, containing both in-plane (parallel to the surface) and out-of-plane (perpendicular to the surface) components, is defined as

d₃₃=(e₃₃(c₁₁+c₁₂)−2e₃₁c₁₃)(c₃₃(c₁₁+c₁₂)−2c₁₃²)⁻¹ (1)

where the eᵢⱼ and cᵢⱼ terms represent piezoelectric and elastic coefficients, respectively. In the wurtzite crystal structure, however, the in-plane terms are mostly negligible and serve only to slightly increase the modulus. Thus, an approximate form, including only the out-of-plane components, is commonly used as an effective lower bound, such that

$${d}_{33} ∼ \frac{{e}_{33}}{{c}_{33}} . \qquad ( 2 )$$

The e₃₃ piezoelectric coefficient can be obtained directly from density functional perturbation theory (DFPT). Bernardini et al. defined this piezoelectric coefficient as the sum of two components: one describing the response of the electric field around static ions and the other containing the effect of ionic displacements within the lattice, such that

$${e}_{33} = {e}_{33}^{static - ion} + \frac{4 {eZ}_{33}^{*}}{\sqrt{3} {a}^{2}} \frac{\partial u}{\partial ϵ} . \qquad ( 3 )$$

See F. Bernardini et al. Phys. Rev. B 56, R10024 (1997).

The first term, representing the static-ion component, describes the change in total polarization along the anisotropic c-axis direction in response to strain, with fixed relative ion coordinates. The contribution from this term is generally fairly small and often negative. The second term in Eq. (3) encapsulates the ionic component, where a is the in-plane lattice constant, e is the elementary electron charge, Z*₃₃ is the c-axial component of the Born effective charge tensor, u is the internal lattice parameter describing the separation of cation and anion planes, and ε is applied strain in the c-axial direction.

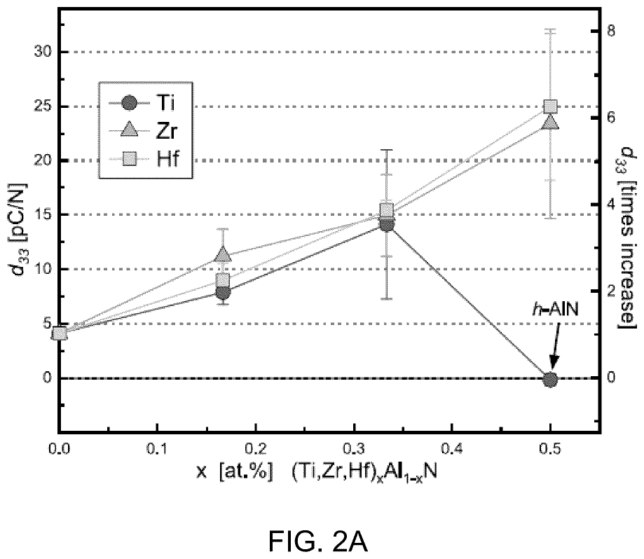

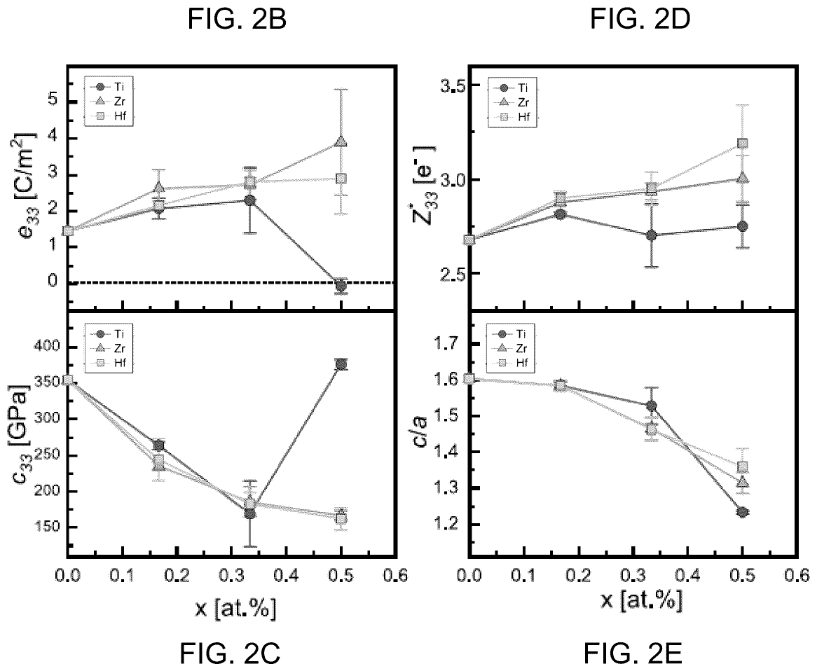

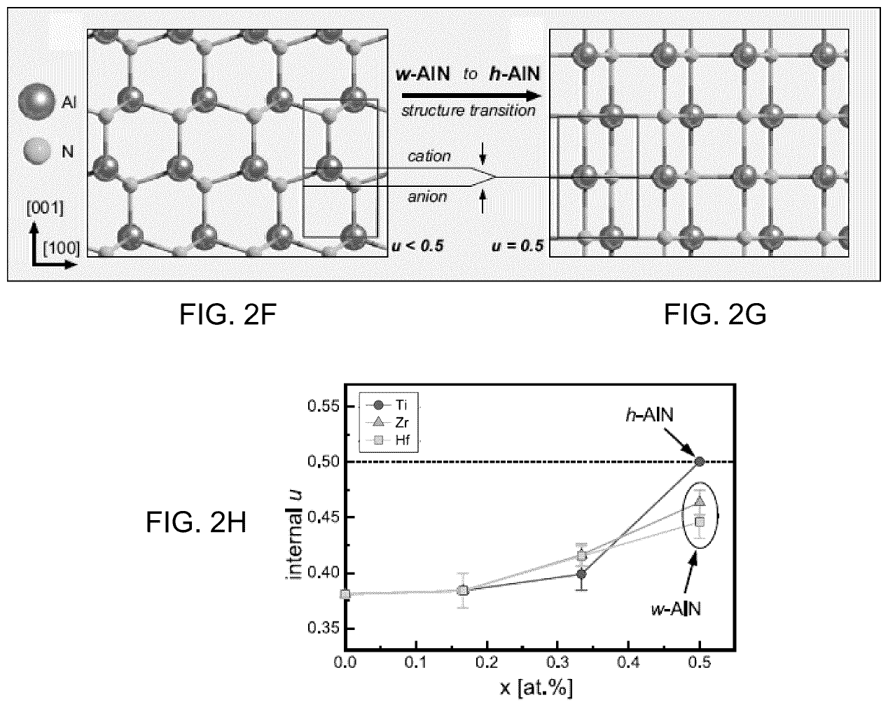

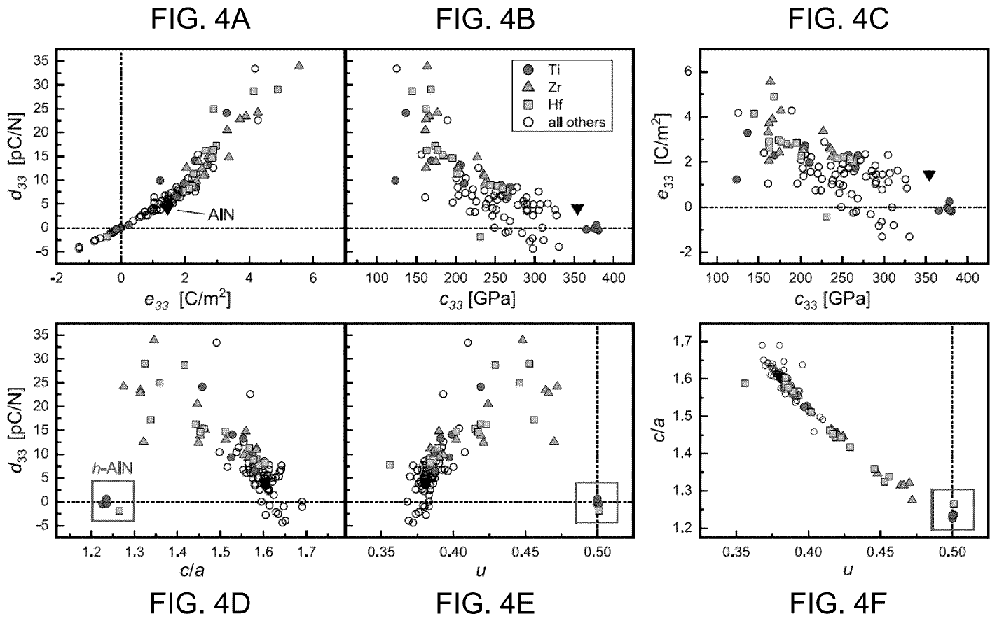

The Born effective charge represents a quantitative description of the change in charge polarization due to a finite displacement in the position of an atom within the lattice. In piezoelectric DFPT calculations, individual Born effective charges were obtained for each atom in the system. These charges were further broken down into directional components (forming a tensor), which captured the polarization response to displacement in each principal direction of the unit cell. In hexagonal wurtzite thin films, where strain is only applied in the surface normal direction, only the c-axial (or ij=33) component of the Born effective charge is important, and so only these components are considered. Thus, in FIGS. 2 and 4, the plotted Z*₃₃ charges represent only the c-axial component of the charge tensor. Additionally, each of the plotted Z*₃₃ charges were computed as the average charge of the atoms in the cation planes of the unit cell. In wurtzite structures, the cation and anion sublattices (planes) take on nearly equal but opposite charges, and so the magnitude of the average Born charge of the cation sublattice (i.e., the values reported in FIGS. 2 and 4) is effectively representative of the average magnitude of the negative anion (N-atoms) sublattice. In such a case, the greater the reported Z*₃₃ charge, the greater the charge separation between adjacent cation and anion sublattices.

### Identification of Alternative Dopants

Twenty three different transition metal dopants (including Sc) in wurtzite AlN (w-AlN) across the d-block in the periodic table were screened for the piezoelectric effect. As a result, it was shown that large piezoelectric enhancements (on par with that of Sc- and Yb-doped AlN) can be achieved in AlN thin films doped with group IVB transition metals (i.e., Ti, Zr or Hf). Using density functional theory (DFT), the piezoelectric and elastic coefficients of these doped systems were calculated for several dopant concentrations and these predictions were then validated by experimentally depositing and characterizing Ti—AlN film candidates. In particular, the group IVB transition metal dopants offer promising performance in comparison to Sc and other rare-earth dopants. Key features and descriptors are identified that enable future discovery and optimization of a broad class of piezoelectric compounds.

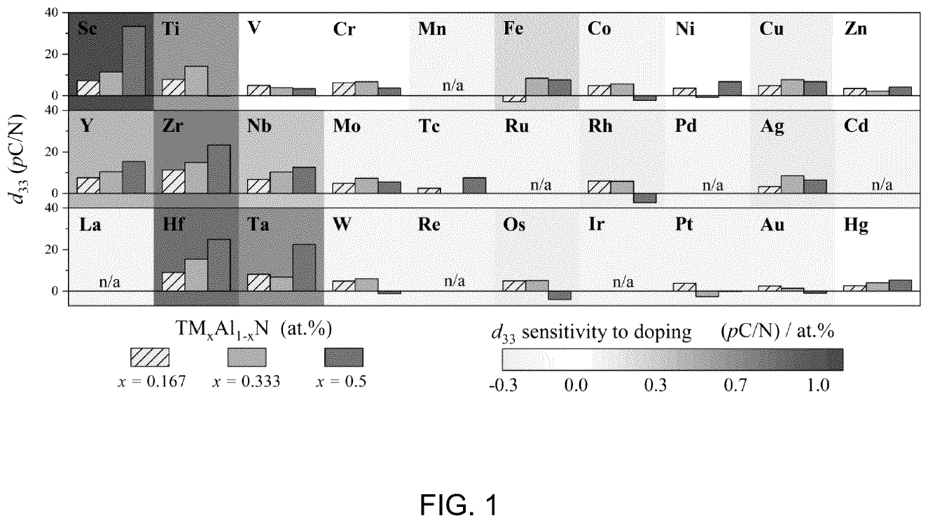

Using density functional perturbation theory (DFPT), the e₃₃ piezoelectric coefficients and c₃₃ elastic constants were calculated to obtain the d₃₃ piezoelectric moduli for doped w-AlN structures, according to Eq. (2), with dopants selected from the full width of the d-block in the periodic table (i.e., 23 out of 30 elements). Transition metal (TM) dopant atoms were randomly substituted into the Al-sublattice of the (TM)ₓAl₁₋ₓN structure at concentrations of x=0.166, 0.333, and 0.5 (under this notation, x=0.5 corresponds to 25 at. % of the whole composition). The piezoelectric moduli of the 23 doped AlN systems along with their relative sensitivity to the dopant (color of the box) are shown in FIG. 1. Piezoelectric enhancements that are comparable to Sc-AlN and Y-AlN films only occur in systems doped with elements belonging to groups III-VB. Specifically, titanium (Ti), zirconium (Zr), and hafnium (Hf) from the group IVB metals produced the largest enhancements to the d₃₃ modulus. To a lesser extent, the group VB metals, niobium (Nb) and tantalum (Ta), also improved the piezoelectric modulus. Beyond these two groups, however, no substantial improvements to the piezoelectric modulus were observed in the d-block TMs.

To better understand the Ti-, Zr-, and Hf-doped structures, and to reduce the uncertainty resulting from finite approximations of random atomic structure, each group IVB composition was modeled four times with random atomic ordering of Al-sublattice (i.e., the dopant and Al atoms were randomly shuffled for each model). The d₃₃ piezoelectric moduli as a function of dopant atom concentration in the Al-sublattice is shown in FIG. 2A. The error bars represent the standard deviation of the four structures at each composition. At concentrations up to x=0.5, Zr and Hf doping produced respective d₃₃ enhancements of 570% and 608%, respectively, relative to the pure w-AlN calculated d₃₃. Ti doping showed a similar behavior up to x=0.333, with an enhancement of 345%; however, further increasing the concentration of Ti resulted in a decrease in d₃₃. This abrupt change is due to structural changes in the wurtzite lattice. For comparison, Akiyama et al. experimentally measured a nearly 500% increase in the d₃₃ modulus for a maximum Sc concentration of x=0.43. See M. Akiyama et al., Adv. Mater. 21, 593 (2009).

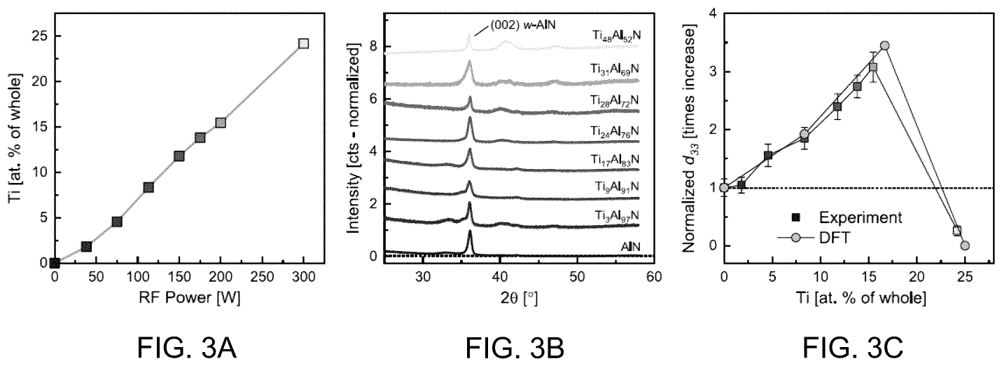

To validate these theoretical predictions, Ti—AlN films were deposited onto high conductivity silicon substrates via magnetron co-sputtering using Ti and Al targets in a nitrogen-rich plasma and their piezoelectric response was measured. See N. Jackson, Vacuum 132, 47 (2016). The thickness of the deposited films was about 500 nm. Ti was chosen in order to capture both the predicted increase and decrease of the piezoelectric response with increasing concentration of Ti. To do so, eight films were deposited; one pure AlN film and the rest of the films with increasing Ti concentrations, with x going up to x=0.484 (energy-dispersive X-ray spectroscopy (EDS) and X-ray diffraction (XRD) characterizations of these thin films are shown in FIGS. 3A and 3B, respectively). The experimentally measured d_{33,f} moduli (where the f subscript denotes measurement of the ‘clamping film’) of the fabricated TiₓAl₁₋ₓN thin films are presented in FIG. 3C alongside the theoretical DFT moduli, and normalized to show the change in piezoelectric response relative to that of the pure AlN compound. The theoretical predictions and measured piezoelectricity moduli are in good agreement, including the steep drop-off between x=0.33 and x=0.5. These experimental results confirm that the doping element is important to achieve good piezoelectric performance and that there is an optimal concentration for which such performance can be achieved. Indeed, as predicted by the DFPT calculations, the experiments confirm that for high concentrations of Ti (i.e., concentrations above 20 at %), the d₃₃ modulus drops substantially as compared to the pure, undoped AlN piezoelectric performance. These measurements also demonstrate the ease of fabrication of group IVB metal doped w-AlN thin films, and suggest that chemically similar Zr and Hf would have comparable results.

### Descriptors of Piezoelectric Performance

To break down the origin of piezoelectric enhancements due to transition metal dopants, the Ti, Zr, and Hf-doped AlN systems were investigated in more detail. FIGS. 2B-2H show the individual component contributions to the piezoelectric d₃₃ modulus (see Eqs. (2) and (3)). FIGS. 2B and 2C indicate a synergistic effect occurring in the w-AlN lattice when doped with Ti, Zr or Hf. In this case, the d₃₃ enhancement is driven by a simultaneous increase in the piezoelectric e₃₃ coefficient (FIG. 2B) accompanied by a softening of the elastic c₃₃ coefficient (FIG. 2C). This combined effect was also observed in the Sc-AlN system by Tasnadi et al. and demonstrates that the piezoelectric polarization is intrinsically connected to the structural stiffness and stability of the wurtzite structure. See F. Tasnadi et al., Appl. Phys. Lett. 94, 151911 (2009). In a later work, Tasnadi et al. showed that this structural weakening (or ‘lattice frustration’), can be ascribed to a partial transition of the wurtzite structure into a theorized hexagonal (h-AlN) structure. See F. Tasnadi et al., Phys. Rev. Lett. 104, 137601 (2010). In this structural transition, the unit cell compresses along the c-direction and expands in the a-direction, while internally the coupled cation and anion planes shift closer to one another. The position of the interior planes along the c-axis is described according to an internal lattice parameter u, which describes the fractional position of the bottom of the two planes in the unit cell. FIGS. 2F-2H illustrate this mechanism. Tasnadi et al. explained that this lattice frustration corresponds to a flattening of the energy landscape between the two structures as the concentration of the dopant increases. See F. Tasnadi et al., Phys. Rev. Lett. 104, 137601 (2010). Their explanation was validated in subsequent investigations by Tholander et al., who observed this energy flattening effect in other doped and co-doped AlN systems and linked the difference in energy of the two structures to the overall piezoelectric enhancement. See C. Tholander et al., Phys. Rev. B 87, 094107 (2013); and C. Tholander et al., Phys. Rev. B 92, 174119 (2015).

The same partial structural transition was found in Ti, Zr, and Hf-doped AlN, as illustrated in FIGS. 2E-2H. As the dopant concentration increases, the c/a ratio decreases (resulting both from increases in a and decreases in c lattice constants), while the internal lattice parameter u increases. Once this transition is completed and the structure is fully h-AlN (i.e., u=0.5 and the cation and anion planes lie exactly in line with one another at the center of the unit cell, as shown in FIG. 2G), the spontaneous and piezoelectric polarizations are lost. However, before this full structural change is realized, the piezoelectric modulus d₃₃ increases significantly, as shown in FIG. 2A. The Ti system exemplifies both aspects of this transition. For low concentrations of Ti (x<0.33), the structural transition is not fully completed, as shown in FIGS. 2E and 2H, and piezoelectric enhancement is observed. Conversely, at high concentration of Ti (x=0.5), the structural transition has fully completed (u has shifted to 0.5), such that the system loses its piezoelectricity. Interestingly, the elastic stiffness (c₃₃) also increases greatly at u=0.5, further suggesting the existence of an intrinsic link between piezoelectricity and elasticity.

Looking beyond group IVB dopants, these results show that piezoelectric enhancement is achieved in doped systems which exhibit both high e₃₃ piezoelectric coefficients, as shown in FIG. 4A, and low c₃₃ elastic coefficients, as shown in FIG. 4B. The correlation of these coefficients, shown in FIG. 4C, further confirms the strong coupling effect between them. Across all the 23 dopants surveyed, it was observed that piezoelectric enhancement only occurs in doped structures that move along the w-AlN→h-AlN structural transition path, as shown in FIGS. 4D and 4E. Doped structures that do not move along this path, (i.e., structures that show no significant changes in either c/a or u parameters) do not exhibit any significant or consistent changes in piezoelectricity.

In the absence of a full DFPT calculation (which can be computationally expensive and difficult to perform) these structural parameters (a, c, c/a, and u) can be obtained from a relatively inexpensive structural DFT relaxation simulation. Together, these structural parameters represent easily identifiable features that indicate whether or not a specific dopant will offer a piezoelectric enhancement, while also indicating the magnitude of this enhancement. Thus, these features can serve as descriptors in both classification and regression style models for a data analytics-focused investigation.

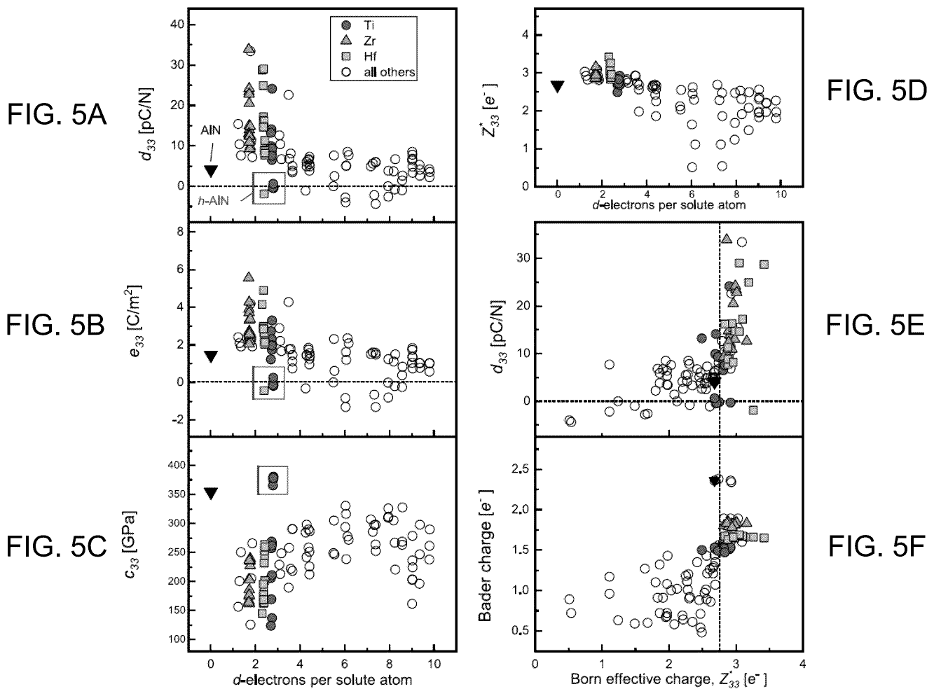

Similar to the structural features discussed above, it was found that simple electronic features of the dopant can also indicate whether a dopant has the potential to enhance piezoelectricity in the w-AlN structure. Dopants that have a low number of d-electrons (≤4) in their valence shell (i.e., groups III-VB) exhibit far greater changes to the piezoelectric modulus and its associated e₃₃ and c₃₃ components than dopants with many (>4) d-electrons, as shown in FIGS. 5A-5C. Dopants with few d-electrons (≤4) all share low electronegativities, particularly in relation to the strong electron affinity of the nitrogen sub-lattice. These weakly electronegative dopants are easily ionized by the nitrogen sub-lattice, resulting in a greater charge difference between alternating cation and anion planes. In turn, this charge difference results in a stronger electric field under piezoelectric polarization.

Likewise, as shown in FIG. 5D, these easily ionizable dopants (primarily from groups III-VB) exhibit the greatest Z*₃₃ Born effective charges (i.e., electrical polarization induced by sublattice displacement). It is also a direct component of the e₃₃ piezoelectric coefficient, as shown in Eq. (3). Piezoelectric enhancements primarily occur for doped structures that exhibit an increase in Z*₃₃ relative to the undoped w-AlN structure, as shown in FIG. 5E. However, because DFPT calculations are computationally cumbersome, the use of Z*₃₃ charges as descriptors is not ideal for high-throughput investigations. Instead, an effective Bader charge (i.e., the average charge of a dopant atom in the w-AlN structure, as counted via a Bader partitioning of the charge density) is a suitable and comparatively easy-to-obtain alternative to Z*₃₃. See M. Yu and D. R. Trinkle, J. Chem. Phys. 134, 064111 (2011). Bader charges are plotted in FIG. 5F as a function of Z*₃₃, and they can be directly compared to d₃₃ values in FIG. 5E. While there is not an exact correlation between the Born effective charge Z*₃₃ and the Bader charge, there is an overall agreement. As indicated by the dashed vertical line spanning both figures, generally one can assume that a dopant carrying a large Bader charge (>1.5e⁻) is likely to also have a large Born effective charge (>2.7e⁻), which is shown in FIG. 5E, and is closely correlated with the overall piezoelectric performance. This trend is especially illustrated when coloring the Ti, Zr, and Hf doping elements which have the highest d₃₃ predictive values and corresponding highest Bader charges (and equivalently the highest Born effective charges). This relationship demonstrates that Bader charges, which can be obtained rather quickly via the same structural DFT relaxation calculation used to estimate c/a and u, can roughly serve as an efficient classification predictor for piezoelectric enhancement of doped-AlN and other wurtzite structures.

The present invention has been described as a earth-abundant dopants for piezoelectric enhancement in wurtzite crystals. It will be understood that the above description is merely illustrative of the applications of the principles of the present invention, the scope of which is to be determined by the claims viewed in light of the specification. Other variants and modifications of the invention will be apparent to those of skill in the art.

1. A piezoelectric material having a wurtzite crystal structure doped with one or more group IVB or VB transition metal dopants.

2. The piezoelectric material of claim 1, wherein the piezoelectric material comprises TMₓAl₁₋ₓN, where TM is a group IVB or VB transition metal.

3. The piezoelectric material of claim 2, wherein 0.16≤x≤0.5.

4. The piezoelectric material of claim 1, wherein the piezoelectric material comprises AlN.

5. The piezoelectric material of claim 1, wherein the piezoelectric material comprises GaN or ZnO.

6. The piezoelectric material of claim 1, wherein the one of more group IVB transition metal dopants comprises Ti, Zr, or Hf.

7. The piezoelectric material of claim 1, wherein the one of more group VB transition metal dopants comprises Nb or Ta.

8. The piezoelectric material of claim 1, wherein the piezoelectric material is fabricated by co-sputtering of the piezoelectric material with the one or more group IVB or VB transition metal dopants.
